(19) United States
(12) Patent Application Publication
(10) Pub. No.: US 2013/0023792 A1
(43) Pub. Date: Jan. 24, 2013

(54) AUTOMATED BLOOD SAMPLING SYSTEM AND METHOD

(71) Applicants: Brian G. Markey, Park Forest, IL (US); Michael G. Lowery, Wildwood, IL (US)

(72) Inventors: Brian G. Markey, Park Forest, IL (US); Michael G. Lowery, Wildwood, IL (US)

(21) Appl. No.: 13/552,868

(22) Filed: Jul. 19, 2012

Related U.S. Application Data
(60) Provisional application No. 61/509,783, filed on Jul. 20, 2011.

(51) Int. Cl. A61B 5/157

(52) U.S. Cl. 600/578

(57) ABSTRACT

Automated blood sampling systems and methods of use are disclosed which are configured to withdraw and test the blood of a mammal without clotting of the withdrawn blood in a main flow path of the automated blood sampling system. The automated blood sampling systems and methods do not require the use of an anti-blood-clotting solution or drug such as Heparin to avoid clotting of the blood in the main flow path of the automated blood sampling system.

### FIELD OF THE INVENTION

The present invention relates to automated blood sampling systems and methods of use which are configured to withdraw and test blood of a mammal while avoiding clotting of the withdrawn blood in a main flow path of the automated blood sampling system without the necessity of using an anti-blood-clotting solution or drug such as Heparin.

### BACKGROUND OF THE INVENTION

Systems and methods exist for withdrawing and testing the blood of mammals. Some of the existing systems and methods keep the withdrawn blood stagnant in a main flow path of the system for a significant amount of time while the withdrawn blood is being tested. These systems typically use anti-blood-clotting solutions, such as Heparin, to inhibit the withdrawn blood from clotting in the main flow path of the blood sampling system. The use of anti-blood-clotting solutions increases cost, and may lead to patient complications. Other existing systems and methods use complex configurations to withdraw and test the blood of mammals while avoiding clotting of the withdrawn blood. This complexity leads to increased cost.

A simple system and method of use is needed to withdraw and test the blood of a mammal without the clotting of the withdrawn blood in the system, and while avoiding one or more of the issues with one or more of the existing systems and methods.

### SUMMARY OF THE INVENTION

In one embodiment, a blood sampling system includes a storage device, a main flow path, a testing flow path, a testing device, at least one valve, and at least one pump. The storage device is configured to contain fluid. The main flow path is configured to flow blood, from a mammal, into the main flow path. The testing flow path is connected to the main flow path. The testing device is disposed in the testing flow path for testing the blood. The at least one valve is configured to toggle between a first setting in which the main flow path is open and the testing flow path is closed, and a second setting in which the main flow path is closed and the testing flow path is open. The at least one pump is configured to: pump the blood along the main flow path when the at least one valve is in the first setting and the blood is not disposed in the testing flow path; pump the blood from the main flow path into and along the testing flow path when the at least one valve is in the second setting; and pump the fluid along a reverse of the main flow path, to flush the blood out of the main flow path, when the at least one valve is in the first setting and the blood is disposed in the testing flow path. The at least one valve is configured to toggle between the first setting and the second setting to prevent the blood from clotting along the main flow path.

In another embodiment, a method of sampling blood without clotting is disclosed. In one step, a blood sampling system is provided. The blood sampling system includes at least one pump, at least one valve, a main flow path, a testing flow path connected to the main flow path, a testing device, and a storage device. In another step, blood from a mammal is pumped, using the at least one pump, into and along the main flow path while the at least one valve is disposed in a first setting, in which the main flow path is open and the testing flow path is closed, and while the blood is not disposed in the testing flow path. In yet another step, the at least one valve is toggled to a second setting, in which the main flow path is closed and the testing flow path is open, and the blood from the main flow path is pumped, using the at least one pump, into and along the testing flow path to the testing device. In still another step, the at least one valve is toggled to the first setting while the blood is disposed in the testing flow path, and fluid from the storage device is pumped, using the at least one pump, along a reverse of the main flow path to flush the blood out of the main flow path before the blood in the main flow path has time to clot along the main flow path. In an additional step, the testing device tests the blood in the testing flow path.

In yet another embodiment, a blood sampling system includes a storage device, a main flow path, a testing flow path, a testing device, at least one valve, and at least one pump. The storage device is configured to contain fluid. The main flow path is configured to flow blood, from a mammal, into the main flow path. The testing flow path is connected to the main flow path. The testing device is disposed in the testing flow path for testing the blood. The at least one valve is configured to toggle between a first setting in which the main flow path is closed and the testing flow path is open, and a second setting in which the main flow path is open and the testing flow path is closed. The at least one pump is configured to: pump the blood to be tested from the mammal into the main flow path and into and along the testing flow path to the testing device when the at least one valve is in the first setting; pump the fluid along a reverse of the main flow path, to flush the blood out of the main flow path, when the at least one valve is in the second setting and the blood is disposed at the testing device along the testing flow path; and pump the blood out of the testing flow path through the reverse of the testing flow path and through a portion of the reverse of the main flow path when the at least one valve is in the first setting after the blood in the testing flow path has been tested by the testing device. The at least one valve is configured to toggle between the first setting and the second setting to prevent the blood from clotting along the main flow path.

In still another embodiment, a method is disclosed of sampling blood without clotting. In one step, a blood sampling system is provided. The provided blood sampling system includes at least one pump, at least one valve, a main flow path, a testing flow path connected to the main flow path, a testing device, and a storage device. In another step, blood from a mammal is pumped, using the at least one pump, into the main flow path and into and along the testing flow path to the testing device while the at least one valve is disposed in a first setting in which the main flow path is closed and the testing flow path is open. In still another step, the at least one valve is toggled to a second setting, in which the main flow path is open and the testing flow path is closed, while the blood is disposed at the testing device in the testing flow path, and fluid from the storage device is pumped, using the at least one pump, along a reverse of the main flow path to flush the blood out of the main flow path before the blood in the main flow path has time to clot along the main flow path. In yet another step, the blood in the testing flow path is tested using the testing device. In an additional step, the at least one valve is toggled to the first setting, and the tested blood is pumped, using the at least one pump, out of the testing flow path through a reverse of the testing flow path and through a portion of the reverse of the main flow path.

These and other features, aspects and advantages of the disclosure will become better understood with reference to the following drawings, description and claims.

### BRIEF DESCRIPTION OF THE DRAWINGS

FIG. 1 is a pictorial schematic view of one embodiment of an automated blood sampling system for withdrawing and testing the blood of a mammal;

FIG. 2 is a pictorial schematic view of another automated blood sampling system for withdrawing and testing the blood of a mammal;

FIG. 3 is a flowchart illustrating a method of automatically withdrawing and testing the blood of a mammal without clotting;

FIG. 4 is a pictorial schematic view of yet another automated blood sampling system for withdrawing and testing the blood of a mammal; and

FIG. 5 is a flowchart illustrating another method of automatically withdrawing and testing the blood of a mammal without clotting.

### DETAILED DESCRIPTION OF THE DISCLOSURE

The following detailed description includes the best currently contemplated mode of carrying out the invention. The description is not to be taken in a limiting sense, but is made merely for the purpose of illustrating the general principles of the invention, since the scope of the invention is best defined by the appended claims. It is noted that the Figures are purely for illustrative purposes and are not to scale.

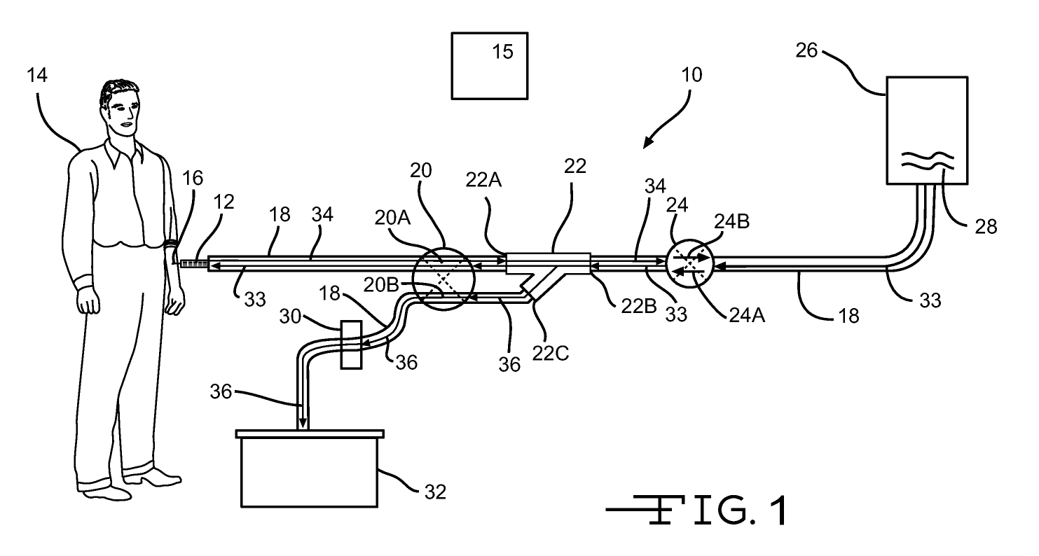

FIG. 1 illustrates an automated blood sampling system 10 for withdrawing and testing the blood 12 of a mammal 14. The automated blood sampling system 10 comprises: a processor/controller 15; a blood removal device 16; tubing 18; valve 20; tubing connector 22; pump 24; storage device 26 containing a fluid 28; testing device 30; container 32; main flow path 34; and testing flow path 36. The processor/controller 15 comprises one or more processors or controllers which are adapted to operate the automated blood sampling system 10. In one embodiment, the processor/controller 15 may control the entire automated blood sampling system 10 including the blood removal device 16, the valve 20, the pump 24, the storage device 26, the testing device 30, and the container 32. In another embodiment, the processor/controller 15 may be incorporated into the pump 24 and may be configured to control both the pump 24 and the valve 20. In other embodiments, varied numbers and types of processor/controller 15 may be used to operate any of the components of the automated blood sampling system 10. The blood removal device 16 comprises a device for removing blood 12 from the mammal 14, and for delivering blood 12 or fluid 28, such as an IV fluid, to the mammal 14. The blood removal device 16 comprises a catheter, a cannula, a needle, or other type of blood removal device or device for vascular access. The tubing 18 comprises a plurality of tubes for transferring blood 12 or fluid 28 between the mammal 14, the blood removal device 16, the valve 20, the tubing connector 22, the pump 24, the storage device 26, the testing device 30, and the container 32. In other embodiments, the tubing 18 may comprise any number and type of tubes.

The valve 20 is preferably activated by an electronic signal and could be hydraulic, pneumatic, or electrical. In one embodiment, the valve 20 may comprise an electrical solenoid pinch valve that is configured to squeeze or compress the tubing 18 shut. In other embodiments, the valve 20 may vary. The valve 20 is configured to automatically toggle between a first setting in which the main flow path 34 is open and the testing flow path 36 is closed, and a second setting in which the main flow path 34 is closed and the testing flow path 36 is open. The valve 20 is configured to automatically toggle between the first setting and the second setting in such a manner as to prevent the blood 12 from clotting along the main flow path 34. The term ‘clotting’ is defined as blood forming blood clots. The valve 20 comprises a double-pinch valve with one portion 20A of the valve 20 configured to independently open and close the main flow path 34 and another portion 20B of the valve configured to independently open and close the testing flow path 36. In other embodiments, the valve 20 may comprise any number or type of valves which are configured to open and close the main flow path 34 and the testing flow path 36.

The tubing connector 22 comprises a V or Y-shaped connector having connections 22A, 22B, and 22C at each end for connecting to the tubing 18. In other embodiments, the tubing connector 22 may comprise a varying number of varying shaped connectors configured to connect varying numbers and types of tubes together. The pump 24 is configured to automatically pump the blood 12 or the fluid 28 through the tubing 18 between the mammal 14, the blood removal device 16, the valve 20, the tubing connector 22, the pump 24, the storage device 26, the testing device 30, and the container 32. The pump 24 may comprise a syringe pump or a peristaltic pump. In other embodiments, the pump 24 may comprise any number or type of pumps. The storage device 26 may comprise a bag or other type of storage device. The storage device 26 may contain fluid 28, such as an IV fluid, sensor calibration fluid, or another type of fluid, for being delivered to the mammal 14. The testing device 30 may comprise a glucose sensor or another type of testing device for testing the blood 12 removed from the mammal 14. The testing device 30 may be biocompatible or non-biocompatible. The container 32 may comprise a waste container connected to the testing flow path 36 for disposing of tested blood 12 after it has been tested by the testing device 30.

The main flow path 34 comprises the main path into which the blood 12 removed from the mammal 14 is flowed into and along. The testing device 30 is disposed in the testing flow path 36. The testing flow path 36 comprises a path connected to the main flow path 34 into which the blood 12 from the main flow path 34 is flowed into in order to test the blood 12 with the testing device 30. The testing flow path 36 and the main flow path 34 do not form a continuous loop. In one embodiment, the testing flow path 36 is 10 inches in length, and the main flow path 34 is 6 inches in length. In other embodiments, the testing flow path 36 is in a range of 1 to 36 inches in length, and the main flow path 34 is in a range of 1 to 18 inches in length. In still other embodiments, the lengths of the testing flow path 36 and the main flow path 34 may vary.

In use, prior to sampling the blood 12 while the valve 20 is in the first setting in which the main flow path 34 is open and the testing flow path 36 is closed, the pump 24 automatically exerts a force in direction 24A to provide a Keep-Vein-Open (KVO) infusion of the fluid 28 from the storage device 26, to the mammal 14, through tubing 18 along the reverse 33 of the main flow path 34. The reverse 33 of the main flow path 34 runs from the storage device 26, through the pump 24, through tubing connections 22B and 22A of tubing connector 22, through one portion 20A of the valve 20, through the blood removal device 16, and to the mammal 14. During this Keep-Vein-Open infusion, portion 20A of the valve 20 is kept open to allow the fluid 28 to run through portion 20A of the valve 20. However portion 20B of the valve 20 is kept closed to stop the fluid 28 after it runs through tubing connections 22B and 22C to prevent the fluid 28 from running through portion 20B of the valve 20 to the testing device 30.

The pump 24 is then automatically reversed, while the valve 20 is still disposed in the first setting in which the main flow path 34 is open and the testing flow path 36 is closed, to exert a pumping force in direction 24B so that blood 12 is withdrawn from a vein of the mammal 14, and drawn through the tubing 18, along the main flow path 34. At this time, blood 12 is not yet disposed in the testing flow path 36. The main flow path 34 runs from the mammal 14, through portion 20A of the valve 20, through tubing connections 22A and 22B of tubing connector 22, towards pump 24. During this step, portion 20A of the valve 20 is kept open and portion 20B of the valve 20 is kept closed to prevent the withdrawn blood 12 from reaching the testing device 30. The withdrawn blood 12 is not allowed to enter the storage device 26. Preferably, 0.5 ml of blood 12 is withdrawn from the mammal 14. In other embodiments, varying amounts of blood 12 may be withdrawn from the mammal 14 in order to obtain an undiluted blood sample past tubing connection 22B.

The valve 20 is then automatically toggled to the second setting in which portion 20A of the valve 20 closes to close the main flow path 34 and in which portion 20B of the valve 20 opens to open the testing flow path 36. This prevents the withdrawn blood 12 located past the tubing connection 22B from flowing back to the mammal 14 through closed portion 20A of the valve 20. The pump direction of the pump 24 is automatically reversed to exert a pumping force along direction 24A. Approximately 0.25 ml of the withdrawn blood 12, which is located past the tubing connection 22B, is then pumped through the tubing 18 from the main flow path 34 into and along the testing flow path 36. During this step the withdrawn blood 12 is pumped from the main flow path 34, through tubing connections 22B and 22C of tubing connector 22, through portion 20B of the valve 20, and into and along the testing flow path 36 to the testing device 30. In other embodiments, a range of 0.1 to 1 ml of the blood 12 may be pumped from the main flow path 34 into and along the testing flow path 36.

The valve 20 is then automatically toggled again to the first setting in which portion 20A of the valve 20 opens to open the main flow path 34 and in which portion 20B of the valve 20 closes to close the testing flow path 36. This prevents withdrawn blood 12 located in the testing flow path 36 from flowing back through portion 20B of the valve 20. The pump 24, which is still exerting the pumping force along direction 24A, is then reset manually or via its automated programming to the Keep-Vein-Open status. The pump 24 then automatically flushes fluid 28 and any remaining withdrawn blood 12 located along main flow path 34 outside of the testing flow path 36 back into the vein of the mammal 14 through tubing 18 along the reverse 33 of the main flow path 34. In such manner, the fluid 28 and any remaining withdrawn blood 12 located along the main flow path 34 is pumped through tubing connections 22B and 22A of the tubing connector 22, through one portion 20A of the valve 20, through the blood removal device 16, and to the mammal 14. While this is taking place, the 0.25 ml of blood 12 to be tested remains in the closed testing path 36 and the testing device 30 automatically tests the withdrawn blood 12 in the testing flow path 36. Preferably approximately 0.5 ml of withdrawn blood 12 and fluid 28 is flushed back into the vein of the mammal 14 during the testing of the blood 12 in the closed testing path 36.

During the entire automated blood sampling process, due to the valve 20 automatic toggling between the first and second settings, the withdrawn blood 12 in the main flow path 34 outside of the testing flow path 36 is only standing stagnant in the main flow path 34 for less than 5 seconds. This avoids clotting of the withdrawn blood 12 in the main flow path 34 without using an anti-blood-clotting solution or drug such as Heparin which in turn reduces cost and avoids potential complications due to the use of an anti-blood-clotting solution. In other embodiments, due to the valve 20 automatic toggling between the first and second settings, the withdrawn blood 12 in the main flow path 34 outside of the testing flow path 36 is only standing in the main flow path 34 for a range of 1 to 60 seconds.

The testing device 30 automatically tests the 0.25 ml of withdrawn blood 12 in the testing flow path 36 for the required analysis time which is approximately 30 seconds. In other embodiments, the testing time and amounts of tested withdrawn blood 12 may vary. For example, in another embodiment the testing time is in the range of 5 to 60 seconds. The valve 20 is then automatically toggled again to the second setting in which portion 20A of the valve 20 closes the main flow path 34 and portion 20B of the valve 20 opens the testing flow path 36. The tested withdrawn blood 12 in the testing flow path 36 is then automatically flushed past the testing device 30 and into the container 32 due to the pump 24 exerting a pumping force along direction 24A. During this step, the pump 24 may pump the fluid 28 to flush the withdrawn blood 12 in the testing flow path 36 out of the testing flow path 36 into the container 32. In other embodiments, the pump 24 may pump the withdrawn blood 12 out of the testing flow path 36 into the container 32 without pumping the fluid 28. Since there is no risk to the vein of the mammal 14 during this stage the flush can be aggressive and conducted at a higher rate (ml/hr) and/or with greater force or fluid pressure. The tested withdrawn blood 12 in the container 32 may then be disposed of according to safe and well known medical procedures. Only approximately 0.25 ml of withdrawn blood 12 is lost during the process. In other embodiments, the amount of withdrawn blood 12 which is lost during the process may vary.

The valve 20 is then automatically toggled again to the first setting in which portion 20A opens to open the main flow path 34 and portion 20B closes to close the testing flow path 36 is closed. The pump 24 then automatically exerts a force in direction 24A providing a Keep-Vein-Open (KVO) infusion of the fluid 28 from the storage device 26 to the mammal 14 through the tubing 18 along the reverse 33 of the main flow path 34. The fluid 28 is infused from the storage device 26, through pump 24, through tubing connections 22B and 22A of tubing connector 22, through one portion 20A of the valve 20, through the blood removal device 16, and into the vascular system of the mammal 14. During this Keep-Vein-Open infusion, portion 20A of the valve 20 is kept open to allow the fluid 28 to run through portion 20A of the valve 20. However, portion 20B of the valve 20 is kept closed to stop the fluid 28 after it runs through tubing connections 22B and 22C to prevent the fluid 28 from running through portion 20B of the valve 20 to the testing device 30.

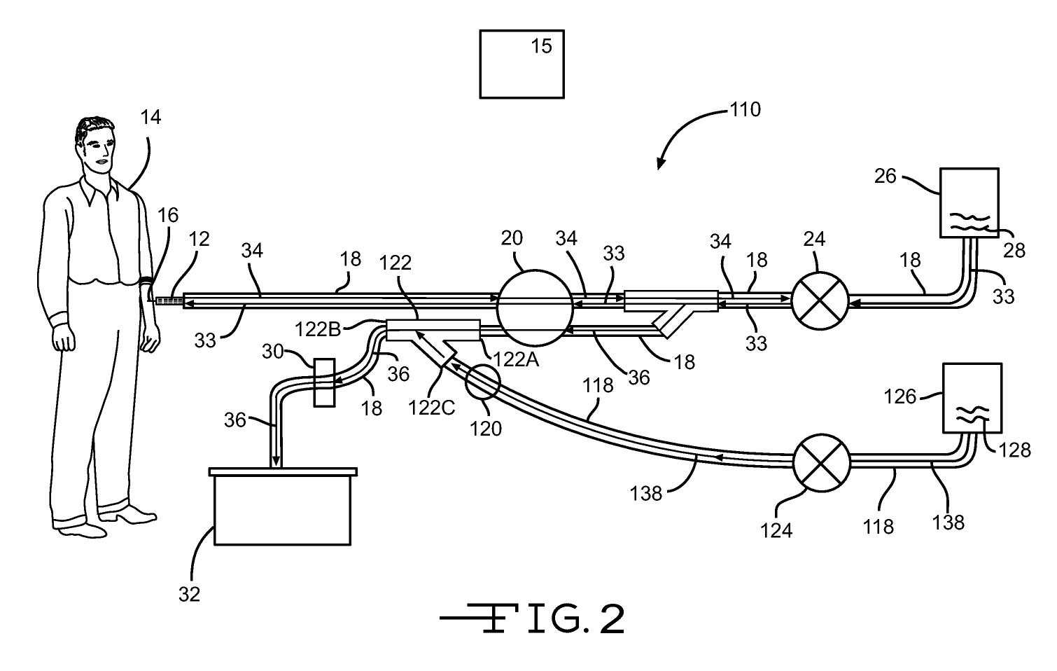

FIG. 2 illustrates another automated blood sampling system 110 for withdrawing and testing the blood 12 of a mammal 14. The automated blood sampling system 110 is identical to the automated blood sampling system 10 of FIG. 1 with the exception of additionally comprising: additional tubing 118; a second valve 120; a second tubing connector 122; a second pump 124; a second storage device 126 containing a second fluid 128; and a flushing flow path 138. The processor/controller 15 may control one or more portions of the automated blood sampling system 10 including the blood removal device 16, the valve 20, the pump 24, the storage device 26, the testing device 30, the container 32, the second valve 120, the second pump 124, and the second storage device 126. The additional tubing 118 comprises tubing for transferring the second fluid 128 along the flushing flow path 138 from the second storage device 126, through the second pump 124, through the second valve 120, through the second tubing connector 122, along the testing flow path 36, over the testing device 30, and into the container 32. The valve 120 is configured to automatically toggle between a setting in which the flushing flow path 138 is closed and another setting in which the flushing flow path 138 is open. The valve 120 comprises a single pinch valve. In other embodiments, the valve 120 may comprise any number or type of valves which are configured to open and close the flushing flow path 138.

The second tubing connector 122 comprises a V or Y-shaped connector having connections 122A, 1228, and 122C at each end for connecting to the tubing 18 and to the additional tubing 118 in order to connect the flushing flow path 138 to the testing flow path 36. In other embodiments, the second tubing connector 122 may comprise a varying number of varying shaped connectors configured to connect varying numbers and types of tubes together. The second pump 124 comprises a syringe pump or a peristaltic pump. In other embodiments, the second pump 124 may comprise any number or type of pumps. The second storage device 126 may comprise a bag or other type of storage device. The storage device 126 may contain second fluid 128, such as a flushing fluid, a buffer solution, or another type of second fluid for flushing over the testing device 30 after the testing device 30 is used to test the blood 12. The flushing flow path 138 comprises the flushing flow path into which the second fluid 128 is flowed through when the second pump 124 pumps the second fluid 128. The flushing flow path 138 is connected to the testing flow path 36.

The automated blood sampling system 110 functions identically to the automated blood sampling system 10 of FIG. 1 with the exception of its additional components. The second valve 120 is kept closed during usage of the automated blood sampling system 110 until after the blood 12 has been tested by the testing device 30, the blood 12 has been pumped into the container 32 by the pump 24, and the valve 20 has been toggled back to its first setting and the pump 24 has exerted a Keep-Vein-Open (KVO) infusion of the fluid 28 from the storage device 26 to the mammal 14 through the tubing 18 along the reverse 33 of the main flow path 34. At this point in time, the testing flow path 36 is closed due to the valve 20 being disposed in the first setting. The second valve 120 is then automatically opened and the second pump 124 automatically pumps the second fluid 128 from the second storage device 126, along the flushing flow path 138, through the open second valve 120, along the connected testing flow path 36, over the testing device 30, and into the container 32. During this step, the testing device 30 is flushed with the second fluid 128. The second valve 120 is then closed and the automated blood sampling system 110 may be reused to test additional blood 12 of the mammal 14.

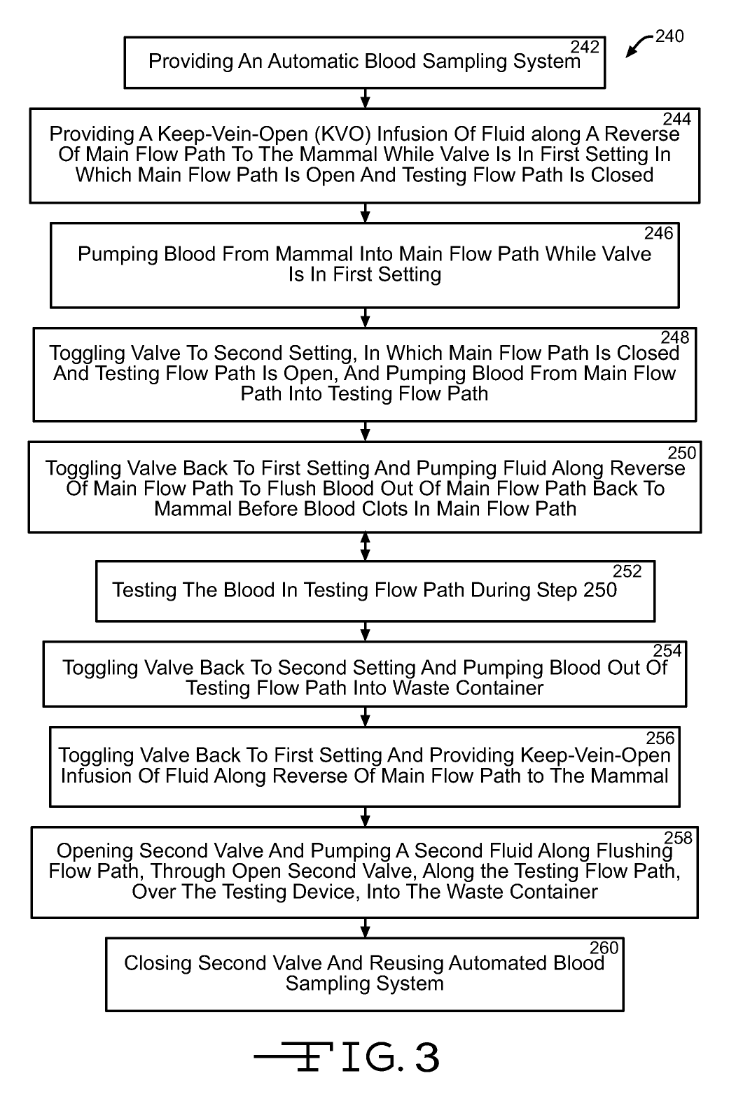

FIG. 3 is a flowchart illustrating a method 240 of automatically withdrawing and testing the blood of a mammal without clotting. As discussed above, at least one processor/controller controls operation of one or more components of the automatic blood sampling system during the steps of the method 240. In step 242, an automatic blood sampling system is provided comprising at least one pump, at least one valve, a main flow path, a testing flow path connected to the main flow path, a testing device, and a storage device. The provided automatic blood sampling system may comprise any of the embodiments disclosed herein. In step 244, while the at least one valve is in a first setting in which the main flow path is open and the testing flow path is closed, the at least one pump automatically provides a Keep-Vein-Open (KVO) infusion of fluid, through tubing, from the storage device to a vein of a mammal along a reverse of the main flow path. In step 246, blood is automatically pumped, using the at least one pump, from the mammal into and along the main flow path while the at least one valve is disposed in the first setting, in which the main flow path is open and the testing flow path is closed, and while the blood is not disposed in the testing flow path.

In step 248, the at least one valve is automatically toggled to a second setting, in which the main flow path is closed and the testing flow path is open, and the blood is pumped, using the at least one pump, from the main flow path into and along the testing flow path to the testing device. During this step, only 0.25 ml of the blood flows from the main flow path into the testing flow path. In other embodiments, the at least one valve may automatically toggle between the first setting and the second setting so that only a range of 0.1 to 1 ml of the blood flows from the main flow path into the testing flow path. In step 250, the at least one valve is automatically toggled to the first setting while the blood is disposed in the testing flow path and fluid is pumped, using the at least one pump, from the storage device along the reverse of the main flow path to flush the blood out of the main flow path back to the vein of the mammal before the blood in the main flow path has time to clot along the main flow path. During the automatically toggling steps, the blood only remains stagnant along the main flow path for less than 5 seconds which prevents clotting of the blood in the main flow path. In other embodiments, the blood may remain stagnant along the main flow path for a range of 1 to 60 seconds to prevent blood from clotting in the main flow path.

In step 252, the blood in the testing flow path is automatically tested using the testing device. The blood in the testing flow path is automatically tested while the at least one valve is in the first setting and while the at least one pump is pumping the fluid from the storage device along the reverse of the main flow path to flush the blood out of the main flow path before the blood in the main flow path has time to clot along the main flow path. The method prevents the blood from clotting along the main flow path without the use of a drug for reducing blood clotting. In step 254, the at least one valve is toggled to the second setting and blood is automatically pumped, using the at least one pump, out of the testing flow path into a waste container after the blood has been tested by the testing device. Only 0.25 ml of the withdrawn blood is lost during this process. In other embodiments, the amount of blood lost during the process may vary.

In step 256, the valve is then automatically toggled again to the first setting, in which the main flow path is open and the testing flow path is closed, and the pump then automatically provides a Keep-Vein-Open (KVO) infusion of the fluid from the storage device to the vein of the mammal through the tubing along the reverse of the main flow path. In step 258, a second valve is then automatically opened and a second pump automatically pumps a second fluid from a second storage device, along a flushing flow path, through an open second valve, along the testing flow path, over the testing device, and into the container. During this step, the testing device is flushed with the second fluid. This flush may be very aggressive since the vein of the mammal is not exposed to the flush. In step 260, the second valve is then closed and the automated blood sampling system may be reused to test additional blood. In alternative embodiments, one or more steps may be added to the method, or one or more steps of the method may be modified.

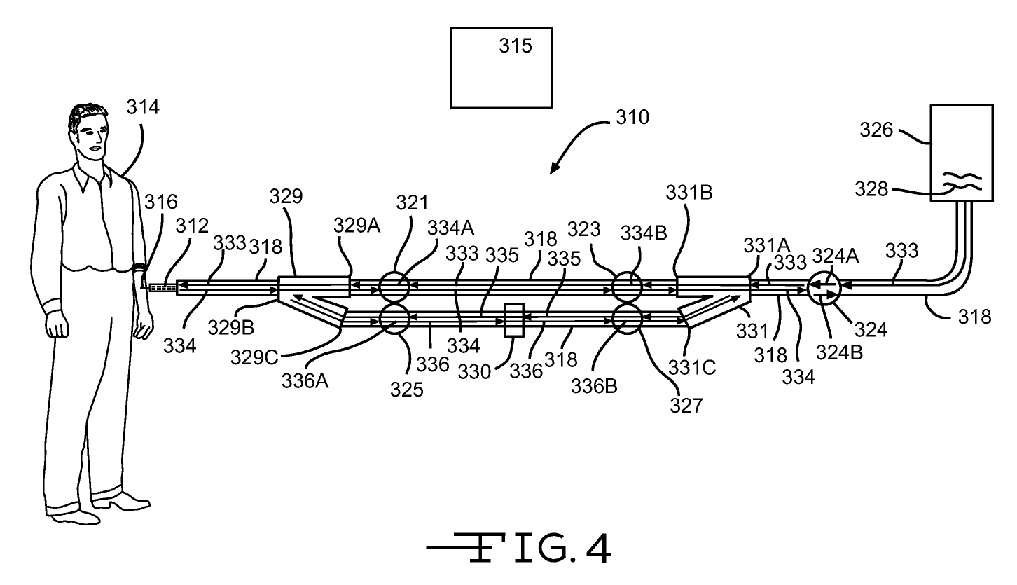

FIG. 4 illustrates another automated blood sampling system 310 for withdrawing and testing the blood 312 of a mammal 314. The automated blood sampling system 310 comprises: a processor/controller 315; a blood removal device 316; tubing 318; valves 321, 323, 325, and 327; tubing connectors 329 and 331; pump 324; storage device 326 containing a fluid 328; testing device 330; main flow path 334; and testing flow path 336. The processor/controller 315 comprises one or more processors or controllers which are adapted to operate the automated blood sampling system 310. In one embodiment, the processor/controller 315 may control the entire automated blood sampling system 310 including the blood removal device 316, the valves 321, 323, 325, and 327, the pump 324, the storage device 326, and the testing device 330. In another embodiment, the processor/controller 315 may be incorporated into the pump 324 and may be configured to control both the pump 324 and the valves 321, 323, 325, and 327. In other embodiments, varied numbers and types of processor/controller 315 may be used to operate any of the components of the automated blood sampling system 310. The blood removal device 316 comprises a device for removing blood 312 from the mammal 314, and for delivering blood 312 or fluid 328, such as an IV fluid, to the mammal 314. The blood removal device 316 comprises a catheter, cannula, needle, or other type of blood removal device. The tubing 318 comprises a plurality of tubes for transferring blood 312 or fluid 328 between the mammal 314, the blood removal device 316, the tubing connectors 329 and 331, the valves 321, 323, 325, and 327, the testing device 330, the pump 324, and the storage device 326. In other embodiments, the tubing 318 may comprise any number of tubes.

The valves 321, 323, 325, and 327 comprise four single pinch valves. Valves 321 and 323 are adapted to independently open and close in order to open and close opposite sides 334A and 334B of the main flow path 334. Valves 325 and 327 are adapted to independently open and close in order to open and close opposite sides 336A and 336B of the testing flow path 336. The valves 321, 323, 325, and 327 are configured to automatically toggle between a first setting in which the main path 334 is closed and the testing path 336 is open, a second setting in which the main path 334 is open and the testing path 336 is closed, and a third setting in which the main path 334 is open and the testing path 336 is open. The valves 321, 323, 325, and 327 are configured to automatically toggle between the first, second, and third settings in such a manner as to prevent the blood 312 from clotting along the main flow path 334. In other embodiments, the valves 321, 323, 325, and 327 may comprise any number or types of valves for opening and closing the main flow path 334 and the testing flow path 336 such as two double-pinch valves, each connected to both the main flow path 334 and the testing flow path 336, or other numbers and types of valves.

The tubing connectors 329 and 331 comprise two V or Y-shaped connectors. Tubing connector 329 has connections 329A, 329B, and 329C at each end for connecting to the tubing 318. Similarly, tubing connector 331 has connections 331A, 331B, and 331C at each end for connecting to the tubing 318. The tubing connectors 329 and 331 join the main flow path 334 and the testing flow path 336 into a continuous loop. In other embodiments, the tubing connectors 329 and 331 may comprise a varying number of varying shaped connectors configured to connect varying numbers and types of tubes together.

The pump 324 is configured to automatically pump the blood 312 or the fluid 328 through the tubing 318 between the mammal 314, the blood removal device 316, the tubing connectors 329 and 331, the valves 321, 323, 325, and 327, the testing device 330, and the storage device 326. The pump 324 may comprise a syringe pump or a peristaltic pump. In other embodiments, the pump 324 may comprise any number or type of pumps. The storage device 326 may comprise a bag or other type of storage device. The storage device 326 may contain fluid 328, such as an IV fluid or another type of fluid, for being delivered to the mammal 314. The testing device 330 may comprise a glucose sensor or another type of testing device for testing the blood 312 removed from the mammal 314. The testing device 330 and the fluid 328 are biocompatible.

The main flow path 334 comprises the main path into which the blood 312 removed from the mammal 314 is flowed into and along. The main flow path 334 runs from the mammal 314, through tubing connections 329B and 329A of tubing connector 329, through valves 321 and 323, through tubing connections 331B and 331A of tubing connector 331, towards pump 324. The reverse 333 of the main flow path 334 runs from the storage device 326, through the pump 324, through tubing connections 331A and 331B of tubing connector 331, through valves 323 and 321, through tubing connections 329A and 329B of tubing connector 329, through the blood removal device 316, and to the vein of the mammal 314.

The testing device 330 is disposed in the testing flow path 336. The testing flow path 336 comprises a path connected to the main flow path 334 into which the blood 312 from the main flow path 334 is flowed into in order to test the blood 312 with the testing device 330. The testing flow path 336 is connected to the main flow path 334 in a continuous loop. The testing flow path 336 runs from tubing connections 329B and 329C of tubing connector 329, through valve 325, over testing device 330, through valve 327, and to tubing connections 331C and 331A of tubing connector 331. The reverse 335 of the testing flow path 336 runs from tubing connections 331A and 331C of tubing connector 331, through valve 327, over testing device 330, through valve 325, through tubing connections 329C and 329B of tubing connector 331, and into a portion of the reverse 333 of the main flow path 334. The testing flow path 336 is 6 inches in length, and the main flow path 334 is 6 inches in length. In other embodiments, the testing flow path 336 is in a range of 2 to 12 inches in length, and the main flow path 334 is in a range of 2 to 12 inches in length. In still other embodiments, the lengths of the testing flow path 336 and the main flow path 334 may vary.

In use, prior to sampling the blood 312 while the valves 321, 323, 325, and 327 are disposed in the third setting in which the main flow path 334 is open and the testing flow path 336 is open, the pump 324 automatically exerts a force in direction 324A to provide a Keep-Vein-Open (KVO) infusion of the fluid 328. The Keep-Vein-Open infusion pumps the fluid 328 through the tubing 318 from the storage device 326 to the mammal 314 along both a reverse 333 of the main flow path 334 and along a reverse 335 of the testing flow path 336. During this Keep-Vein-Open infusion, valves 321 and 323 are kept open to allow the fluid 328 to run through the reverse 333 of the main flow path 334. Similarly, valves 325 and 327 are kept open to allow the fluid 328 to run through the reverse 335 of the testing flow path 336. In other embodiments, during this Keep-Vein-Open infusion, valves 321 and 323 may be kept open to allow the fluid 328 to run through the reverse 333 of the main flow path 334, and valves 325 and 327 may be closed to prevent the fluid 328 from running through the reverse 335 of the testing flow path 336.

The valves 321, 323, 325, and 327 are then automatically toggled to a first setting in which valves 321 and 323 close the main flow path 334, and in which valves 325 and 327 open the testing flow path 336. The pump 324 is then automatically reversed, while the valves 321, 323, 325, and 327 are still disposed in the first setting in which the main flow path 334 is closed and the testing flow path 336 is open, to exert a pumping force in direction 324B. During this time, the blood 312 is withdrawn through the tubing 318 from a vein of the mammal 314 into the main flow path 334. The closed valves 321 and 323 prevent the withdrawn blood 312 from flowing completely through the main flow path 334 causing the blood to flow from the main flow path 334 into and along the testing flow path 336 through tubing connections 329B and 329C of tubing connector 329, though valve 325, and along the testing flow path 336 to the testing device 330. Preferably, 0.5 ml of blood 312 is withdrawn from the mammal 314 into the testing flow path 336. In other embodiments, the valves 321, 323, 325, and 327 are configured to automatically toggle between the first setting and the second setting so that only a range of 0.1 to 5 ml of the blood 312 flows from the main flow path 334 into the testing flow path 336. In still other embodiments, varying amounts of blood 312 may be withdrawn from the mammal 314 into the testing flow path 336 in order to obtain an undiluted withdrawn sample of blood 312 at the testing device 330. The withdrawn blood 312 is not allowed to enter the storage device 326.

When the appropriate amount of withdrawn blood 312 is disposed at the testing device 330 along the testing flow path 336, the valves 321, 323, 325, and 327 are then automatically toggled to the second setting in which valves 325 and 327 are closed to close the testing flow path 336, and in which valves 321 and 323 are opened to open the main flow path 334. The pump 324 then automatically sets to a Keep-Vein-Open (KVO) setting and proceeds to pump the fluid 328 from the storage device 326 along the reverse 333 of the main flow path 334 to flush the blood 312 out of the main flow path 334 before the blood 312 in the main flow path 334 has time to clot along the main flow path 334. Approximately 3 ml of blood 312 and fluid 328 is flushed back into the vein of the mammal 314 through the reverse 333 of the main flow path 334. In other embodiments, varying amounts of blood 312 and fluid 328 may be flushed back into the vein of the mammal 314.

During the entire process, including the parts of the process which follow below, the valves 321, 323, 325, and 327 are configured to automatically toggle between the first, second, and third settings to prevent the blood 312 from clotting along the main flow path 334 due to the blood 312 only standing stagnant in the main flow path 334 for less than 1 second. In other embodiments, during the entire process, including the parts of the process which follow below, the valves 321, 323, 325, and 327 are configured to automatically toggle between the first, second, and third settings so that the blood 312 only remains stagnant in the main flow path 334 for a range of 0.1 to 5 seconds. In still other embodiments, the valves 321, 323, 325, and 327 may be configured to allow the blood 312 to remain stagnant for a varying amount of time designed to prevent the blood 312 from clotting in the main flow path 334. In such manner, the method prevents the blood 312 from clotting in the main flow path 334 without the use of an anti-blood-clotting solution or drug such as Heparin which in turn reduces cost and avoids potential complications due to the use of an anti-blood-clotting solution.

While the valves 321, 323, 325, and 327 are disposed in the second setting and the pump 324 is pumping the fluid 328 from the storage device 326 along the reverse 333 of the main flow path 334 to flush the blood 312 out of the main flow path 334, the testing device 330 automatically tests the withdrawn blood 312 at the testing device 330 in the closed testing flow path 336. The testing device 330 tests the withdrawn blood 312 for approximately 30 seconds. In other embodiments, the testing time may vary. After the required testing time, the valves 321, 323, 325, and 327 automatically toggle to the first setting in which the valves 321 and 323 close the main flow path 334, and in which the valves 325 and 327 open the testing flow path 336. The pump 324 then pumps the fluid 328 from the storage device 326, through a reverse 335 of the testing flow path 336, into and through a portion of the reverse 333 of the main flow path 334, and back into the vein of the mammal 314. This causes the tested blood 312 to be flushed out of the testing flow path 336 back into the vein of the mammal 314. During this time, essentially all of the tested blood 312 is flushed back into the vein of the mammal 314. In such manner, essentially no blood 312 of the mammal 314 is lost during the blood sampling process.

The valves 321, 323, 325, and 327 are then automatically toggled to the third setting in which each of the valves 321, 323, 325, and 327 are opened to open both of the main flow path 334 and the testing flow path 336. The pump 324 is then set to a Keep-Vein-Open (KVO) setting and automatically exerts a force in direction 324A to provide a Keep-Vein-Open (KVO) infusion of the fluid 328 from the storage device 326 to the mammal 314 through tubing 318 along both a reverse 333 of the main flow path 334 and along a reverse 335 of the testing flow path 336. During this Keep-Vein-Open infusion, valves 321 and 323 are kept open to allow the fluid 328 to run through the reverse 333 of the main flow path 334. Similarly, valves 325 and 327 are kept open to allow the fluid 328 to run through the reverse 335 of the testing flow path 336. In other embodiments, during this Keep-Vein-Open infusion, valves 321 and 323 may be kept open to allow the fluid 328 to run through the reverse 333 of the main flow path 334, and valves 325 and 327 may be closed to prevent the fluid 328 from running through the reverse 335 of the testing flow path 336.

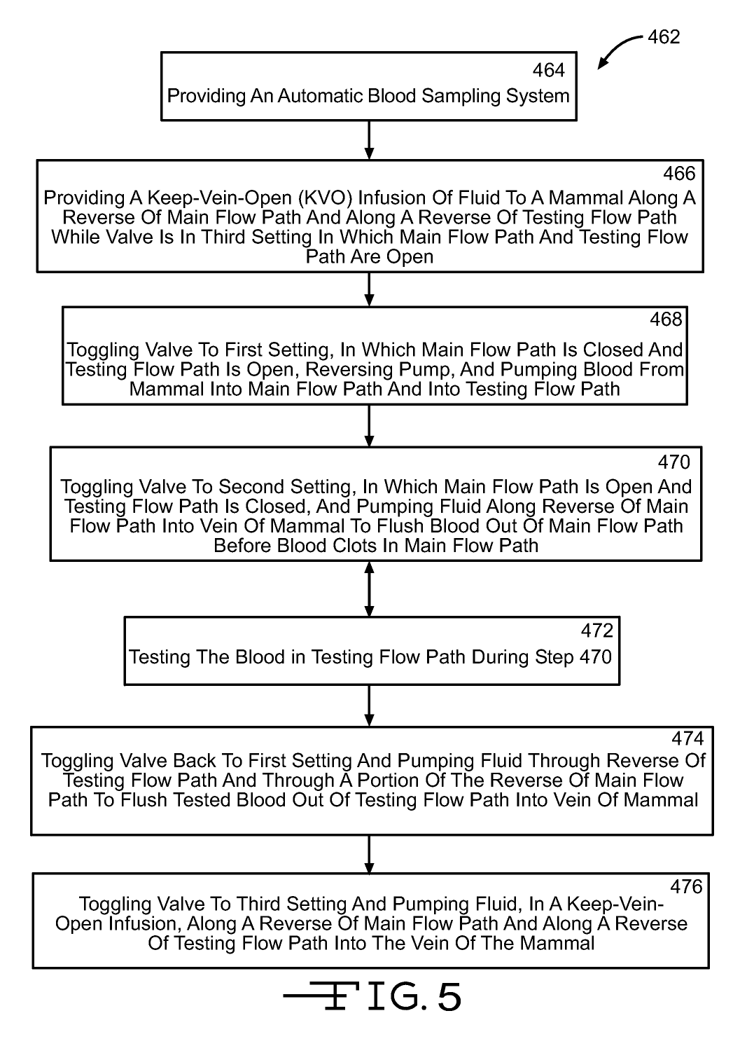

FIG. 5 is a flowchart illustrating another method 462 of automatically withdrawing and testing the blood of a mammal without clotting. As discussed above, at least one processor/controller controls operation of one or more components of the automatic blood sampling system during the steps of the method 462. In step 464, an automatic blood sampling system is provided comprising at least one pump, at least one valve, a main flow path, a testing flow path connected to the main flow path, a testing device, and a storage device. The provided automatic blood sampling system may comprise any of the embodiments disclosed above. In step 466, while the at least one valve is disposed in a third setting in which the main flow path flow path is open and the testing flow path is open, the at least one pump automatically provides a Keep-Vein-Open (KVO) infusion of fluid from the storage device to a vein of a mammal through tubing along a reverse of the main flow path and a reverse of the testing flow path.

In step 468, the at least one valve is toggled to a first setting in which the main flow path is closed and the testing flow path is open, and the at least one pump is reversed to automatically pump blood from the mammal into the main flow path and into and along the testing flow path to the testing device. Due to the toggling of the at least one valve, a range of 0.1 to 5 ml of the blood flows from the main flow path into the testing flow path to be tested by the testing device. In other embodiments, varying amounts of blood may flow into the testing flow path.

In step 470, the at least one valve is automatically toggled to a second setting, in which the main flow path is open and the testing flow path is closed, while the blood is disposed at the testing device in the testing flow path. At this point in time, the at least one pump is set to a Keep-Vein-Open setting and proceeds to pump fluid from the storage device along a reverse of the main flow path into the vein of the mammal to flush the blood out of the main flow path before the blood in the main flow path has time to clot along the main flow path. During this process, the blood is only standing in the main flow path for less than 1 second. In other embodiments, the blood may stay standing in the main flow path for a range of 0.1 to 5 seconds. In step 472, the testing device automatically tests the blood in the testing flow path while the at least one valve is disposed in the second setting and while the at least one pump is pumping the fluid from the storage device along the reverse of the main flow path and into the vein of the mammal during the Keep-Vein-Open setting of step 470.

In step 474, the at least one valve is automatically toggled to the first setting in which the main flow path is closed and the testing flow path is open. At this time, the at least one pump proceeds to pump fluid from the storage device, through a reverse of the testing flow path, through a portion of the reverse of the main flow path, and back into the vein of the mammal in order to flush the tested blood out of the testing flow path back into the vein of the mammal. In step 476, the at least one valve is automatically toggled to the third setting, in which the main flow path flow path is open and the testing flow path is open, and the at least one pump is set to the Keep-Vein-Open (KVO) setting. At this time, the at least one pump infuses the fluid from the storage device, along both a reverse of the main flow path and along a reverse of the testing flow path, to the vein of the mammal. In such manner, no blood of the mammal is lost.

Throughout the method, the at least one valve automatically toggles between the first setting and the second setting so that the blood only remains stagnant along the main flow path for a range of 0.1 to 5 seconds. In such manner, the method prevents the blood from clotting in the main flow path without the use of an anti-blood-clotting solution or drug such as Heparin which in turn reduces cost and avoids potential complications due to the use of an anti-blood-clotting solution. In alternative embodiments, one or more steps may be added to the method, or one or more steps of the method may be modified.

The embodiments of the disclosure allow for the withdrawal and testing of blood from a mammal while avoiding some of the issues associated with one or more of the existing blood sampling systems and methods. For instance, the embodiments of the disclosure avoid the clotting of withdrawn blood in the sampling system using a simple configuration without the requirement of using an anti-blood-clotting solution such as Heparin. This reduces costs and the probability of potential patient complications.

It should be understood, of course, that the foregoing relates to exemplary embodiments of the disclosure and that modifications may be made without departing from the scope of the disclosure as set forth in the following claims. For example, one skilled in the art will appreciate from the above disclosure that an artery or some other portion of the vascular system of a mammal can be accessed with the blood removal device, systems and methods of the present invention.

We claim:

1. A blood sampling system comprising:
a storage device for containing fluid;
a main flow path for flowing blood into from a mammal;
a testing flow path connected to the main flow path;
a testing device disposed in the testing flow path for testing the blood;
at least one valve configured to toggle between a first setting in which the main flow path is open and the testing flow path is closed and a second setting in which the main flow path is closed and the testing flow path is open; and
at least one pump configured to: pump the blood along the main flow path when the at least one valve is in the first setting and the blood is not disposed in the testing flow path; pump the blood from the main flow path into and along the testing flow path when the at least one valve is in the second setting; and pump the fluid along a reverse of the main flow path, to flush the blood out of the main flow path, when the at least one valve is in the first setting and the blood is disposed in the testing flow path;
wherein the at least one valve is configured to toggle between the first setting and the second setting to prevent the blood from clotting along the main flow path.

2. The blood sampling system of claim 1 further comprising a waste container connected to the testing flow path, wherein the at least one pump is configured to pump the blood, after the blood has been tested by the testing device, out of the testing flow path into the waste container while the at least one valve is in the second setting.

3. The blood sampling system of claim 1 wherein the at least one valve is configured to toggle between the first setting and the second setting so that the blood only remains stagnant along the main flow path for a range of 1 to 60 seconds.

4. The blood sampling system of claim 1 wherein the at least one valve is configured to toggle between the first setting and the second setting so that only a range of 0.1 to 1 ml of the blood flows from the main flow path into the testing flow path.

5. The blood sampling system of claim 1 wherein the testing flow path is in a range of 1 to 36 inches long and the main flow path is in the range of 1 to 18 inches long.

6. The blood sampling system of claim 1 wherein the testing device is configured to test the blood disposed along the testing flow path while the at least one valve is in the first setting and the at least one pump is pumping the fluid along the reverse of the main flow path flushing the blood out of the main flow path.

7. The blood sampling system of claim 1 wherein the at least one valve is configured to toggle between the first setting and the second setting to prevent the blood from clotting along the main flow path without the use of a solution for reducing blood clotting.

8. The blood sampling system of claim 1 wherein the main flow path and the testing flow path do not form a continuous loop.

9. The blood sampling system of claim 1 wherein the at least one valve comprises one double pinch valve with one portion of the double pinch valve connected to the main flow path and another portion of the double pinch valve connected to the testing flow path.

10. The blood sampling system of claim 1 further comprising a flushing flow path connected to the testing flow path, a second valve, a second pump, and a second storage device for containing a second fluid, wherein after the blood in the testing flow path is tested by the testing device the second valve is configured to open, and the second pump is configured to pump the second fluid from the second storage device, along the flushing flow path, through the second valve, along the testing flow path, and over the testing device.

11. A method of sampling blood without clotting comprising:
providing a blood sampling system comprising at least one pump, at least one valve, a main flow path, a testing flow path connected to the main flow path, a testing device, and a storage device;
pumping, using the at least one pump, blood from a mammal into and along the main flow path while the at least one valve is disposed in a first setting, in which the main flow path is open and the testing flow path is closed, and while the blood is not disposed in the testing flow path;
toggling the at least one valve to a second setting, in which the main flow path is closed and the testing flow path is open, and pumping, using the at least one pump, the blood from the main flow path into and along the testing flow path to the testing device;
toggling the at least one valve to the first setting while the blood is disposed in the testing flow path and pumping, using the at least one pump, fluid from the storage device along a reverse of the main flow path to flush the blood out of the main flow path before the blood in the main flow path has time to clot along the main flow path; and
testing the blood in the testing flow path using the testing device.

12. The method of claim 11 further comprising toggling the at least one valve to the first setting and pumping, using the at least one pump, the blood out of the testing flow path into a waste container after the blood has been tested by the testing device.

13. The method of claim 11 wherein during the toggling steps the at least one valve toggles between the first setting and the second setting so that the blood only remains stagnant along the main flow path for a range of 1 to 60 seconds.

14. The method of claim 11 wherein during the toggling steps the at least one valve toggles between the first setting and the second setting so that only a range of 0.1 to 1 ml of the blood flows from the main flow path into the testing flow path.

15. The method of claim 11 wherein the testing the blood step occurs while the at least one valve is in the first setting and while the at least one pump is pumping the fluid from the storage device along the reverse of the main flow path to flush the blood out of the main flow path before the blood in the main flow path has the time to clot along the main flow path.

16. The method of claim 11 wherein the method prevents the blood from clotting along the main flow path without the use of a solution for reducing blood clotting.

17. The method of claim 11 further comprising after the blood in the testing flow path is tested by the testing device a second valve opening and a second pump pumping a second fluid from a second storage device, along a flushing flow path, through the second valve, along the testing flow path, and over the testing device.

18. A blood sampling system comprising:
a storage device for containing fluid;
a main flow path for flowing blood into from a mammal;
a testing flow path connected to the main flow path;
a testing device disposed in the testing flow path for testing the blood;
at least one valve configured to toggle between a first setting in which the main flow path is closed and the testing flow path is open and a second setting in which the main flow path is open and the testing flow path is closed; and
at least one pump configured to: pump the blood to be tested from the mammal into the main flow path, and then into and along the testing flow path to the testing device when the at least one valve is in the first setting; pump the fluid along a reverse of the main flow path, to flush the blood out of the main flow path, when the at least one valve is in the second setting and the blood is disposed at the testing device along the testing flow path; and pump the blood out of the testing flow path through the reverse of the testing flow path and through a portion of the reverse of the main flow path when the at least one valve is in the first setting after the blood in the testing flow path has been tested by the testing device;
wherein the at least one valve is configured to toggle between the first setting and the second setting to prevent the blood from clotting along the main flow path.

19. The blood sampling system of claim 18 wherein the main flow path and the testing flow path form a continuous loop, the testing device is biocompatible, and the pump is configured to pump the blood out of the testing flow path and back into the mammal through the reverse of the testing flow path and through the portion of the reverse of the main flow path when the at least one valve is in the first setting after the blood in the testing flow path has been tested by the testing device.

20. The blood sampling system of claim 18 wherein the at least one valve is configured to toggle between the first setting and the second setting so that the blood only remains stagnant along the main flow path for a range of 0.1 to 5 seconds.

21. The blood sampling system of claim 18 wherein the at least one valve is configured to toggle between the first setting and the second setting so that only a range of 0.1 to 5 ml of the blood flows from the main flow path into the testing flow path.

22. The blood sampling system of claim 18 wherein the testing flow path is in a range of 2 to 12 inches long and the main flow path is in the range of 2 to 12 inches long.

23. The blood sampling system of claim 18 wherein the testing device is configured to test the blood disposed at the testing device along the testing flow path while the at least one valve is in the second setting and the at least one pump is pumping the fluid along the reverse of the main flow path.

24. The blood sampling system of claim 18 wherein the at least one valve is configured to toggle between the first setting and the second setting to prevent the blood from clotting along the main flow path without the use of a solution for reducing blood clotting.

25. The blood sampling system of claim 18 wherein the at least one valve comprises a plurality of valves which are disposed along opposite sides of the main flow path and the testing flow path.

26. The blood sampling system of claim 18 wherein the at least one valve comprises two double pinch valves connected to both the main flow path and the testing flow path or four single pinch valves each connected to either the main flow path or the testing flow path.

27. A method of sampling blood without clotting comprising:
providing a blood sampling system comprising at least one pump, at least one valve, a main flow path, a testing flow path connected to the main flow path, a testing device, and a storage device;
pumping, using the at least one pump, blood from a mammal into the main flow path and into, and then along the testing flow path to the testing device while the at least one valve is disposed in a first setting in which the main flow path is closed and the testing flow path is open;
toggling the at least one valve to a second setting, in which the main flow path is open and the testing flow path is closed, while the blood is disposed at the testing device in the testing flow path and pumping, using the at least one pump, fluid from the storage device along a reverse of the main flow path to flush the blood out of the main flow path before the blood in the main flow path has time to clot along the main flow path;
testing the blood in the testing flow path using the testing device; and
toggling the at least one valve to the first setting and pumping, using the at least one pump, the tested blood out of the testing flow path through a reverse of the testing flow path and through a portion of the reverse of the main flow path.

28. The method of claim 27 wherein the main flow path and the testing flow path form a continuous loop, the testing device is biocompatible, and further comprising toggling the at least one valve to the first setting and pumping the tested blood out of the testing flow path and back into the mammal through the reverse of the testing flow path and through the portion of the reverse of the main flow path.

29. The method of claim 27 wherein during the toggling steps the at least one valve toggles between the first setting and the second setting so that the blood only remains stagnant along the main flow path for a range of 0.1 to 5 seconds.

30. The method of claim 27 wherein during the toggling steps the at least one valve toggles between the first setting and the second setting so that only a range of 0.1 to 5 ml of the blood flows from the main flow path into the testing flow path.

31. The method of claim 27 wherein the testing the blood step occurs while the at least one valve is in the second setting and while the at least one pump is pumping the fluid from the storage device along the reverse of the main flow path to flush the blood out of the main flow path before the blood in the main flow path has the time to clot along the main flow path.

32. The method of claim 27 wherein the method prevents the blood from clotting along the main flow path without the use of a solution for reducing blood clotting.
